(19) United States
(12) Patent Application Publication
(10) Pub. No.: US 2011/0303227 A1
(43) Pub. Date: Dec. 15, 2011

(54) SPHERICALLY-SHAPED EAR PLUG AND METHOD FOR FORMING THE SAME

(76) Inventors: Douglas Pick, Aliso Viejo, CA (US); Scott Malkoff, Phoenix, AZ (US)

(21) Appl. No.: 13/160,272

(22) Filed: Jun. 14, 2011

Related U.S. Application Data
(60) Provisional application No. 61/354,346, filed on Jun. 14, 2010.

(51) Int. Cl. A61F 11/08; B29C 43/34

(52) U.S. Cl. 128/864; 264/571

(57) ABSTRACT

A spherically-shaped ear plug is described. The ear-plug is formed of silicone to provide pliable and malleable properties. Additionally, because the ear plug is made of silicone, when it is positioned and depressed into the ear canal, the front end of the ear plug is pushed out and deeper into the ear canal to create a safe seal. Finally, the tacky consistency of silicone creates an airtight/watertight seal between the ear plug and ear canal.

### PRIORITY CLAIM

This is a non-provisional application of U.S. Provisional Application No. 61/354,346, filed on Jun. 14, 2010, entitled, “Spherically-Shaped Ear Plug.”

### BACKGROUND OF THE INVENTION

(1) Field of Invention

The present invention relates to an ear plug and, more particularly, to a spherically-shaped ear plug that is formed of silicone and a method for forming the same.

(2) Description of Related Art

Ear plugs have long been known in the art. An earplug is a device that is meant to be inserted in the ear canal to protect the wearer's ears from loud noises or the intrusion of water, foreign bodies, dust or excessive wind.

Traditional ear plugs are often made of foam and compressed prior to insertion in the ear canal. Upon positioning within the ear canal, the ear plug expands to conform and become affixed within the ear canal. User error often creates problems with existing ear plugs in that the user is unfamiliar with the process of affixing the plug with the user's ear. Additionally, due to the varying anatomy of each user, traditional ear plugs do not conform exactly to the user's ear. While operable for short term usage, such traditional ear plugs can become uncomfortable during long-term use.

Custom molded plugs are recommended for long-term use, since they are more comfortable and gentle to the skin and won't go too far into the ear canal. However, custom molded plugs can be expensive and are not readily available on retail shelves.

Thus, a continuing need exists for a safe ear plug that can easily affix with the user's ear to provide the comfort of a custom molded plug at an affordable price.

### SUMMARY OF INVENTION

A spherically-shaped ear plug is described. The ear-plug is formed of silicone to provide pliable and malleable properties. Additionally, because the ear plug is made of silicone, when it is positioned and depressed into the ear canal, the front end of the ear plug is pushed out and deeper into the ear canal to create a safe seal. The tacky consistency of silicone creates an airtight/watertight seal between the ear plug and ear canal.

Additionally, as can be appreciated by one skilled in the art, the present invention also comprises a method for forming and using the ear plug described herein. For example, the method comprises multiple acts, such as using a cookie cutter to shape an uncured silicone compound, thereby filling the cookie cutter with the silicone. The cookie cutter is then positioned in a slot of a first press, the first press having pins with air holes therethrough. The pins of the first press are then driven through the silicone in the cookie cutter to deposit silicone pills into tray slots of an open tray. Air is forced through the air holes as the pins are driven through the silicone to release the silicone pills from the pins and into the tray slots. A second press having a mold with mold slots is used to press the silicone pills between the tray slots and corresponding mold slots. The second press includes a protective release layer (e.g., nitrile disk) to act as a barrier between the silicon and mold. The release layer is vacuumed into the mold, with the mold and release layer being pressed onto the open tray to form the ear plugs. Finally, the second press is released to cause the release layer to spring to its original shape (i.e., flat shape) and release the ear plugs into the open tray. It should be noted that the tray slots and mold slots can be semi-spherically shaped such that together they form the spherically-shaped ear plug.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 is a left, side-view of a spherically-shaped ear plug;

FIG. 2 is a right, side-view of the spherically-shaped ear plug;

FIG. 3 is a front view of the spherically-shaped ear plug;

FIG. 4 is a rear view of the spherically-shaped ear plug;

FIG. 5 is a top view of the spherically-shaped ear plug;

FIG. 6 is a bottom view of the spherically-shaped ear plug;

FIG. 7 is a side-view illustration of the ear plug as positioned against an ear canal;

FIG. 8 is a side-view illustration of the ear plug as being pushed into an ear canal; and

FIG. 9 is a flow chart illustrating a general procedure for forming the ear plug of the present invention.

### DETAILED DESCRIPTION

The present invention relates to an ear plug and, more particularly, to a spherically-shaped ear plug that is formed of silicone and a method for forming the same. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is only one example of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Please note, if used, the labels left, right, front, back, top, bottom, forward, reverse, clockwise and counter clockwise have been used for convenience purposes only and are not intended to imply any particular fixed direction. Instead, they are used to reflect relative locations and/or directions between various portions of an object.

(1) Description

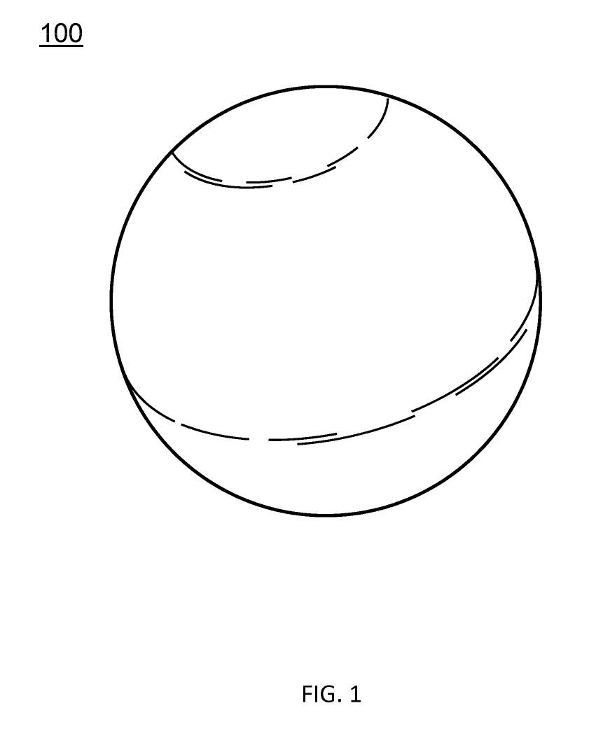

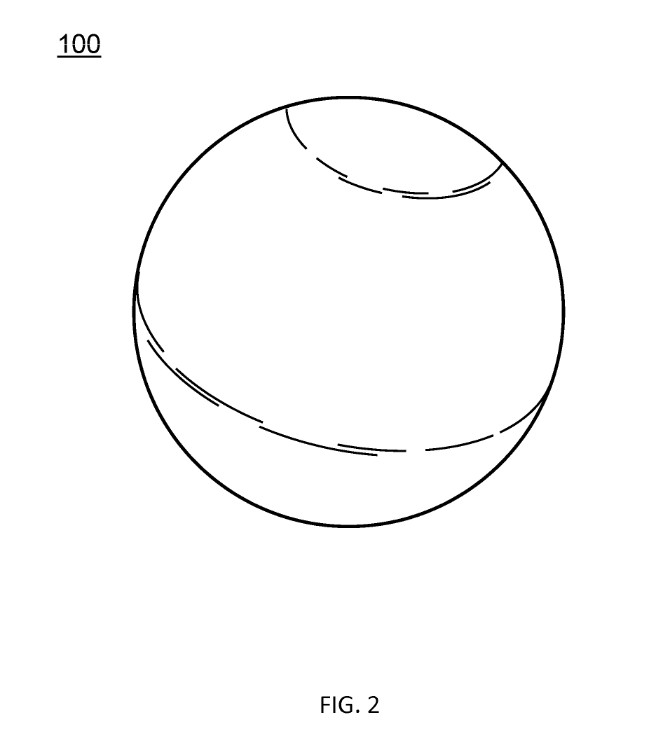

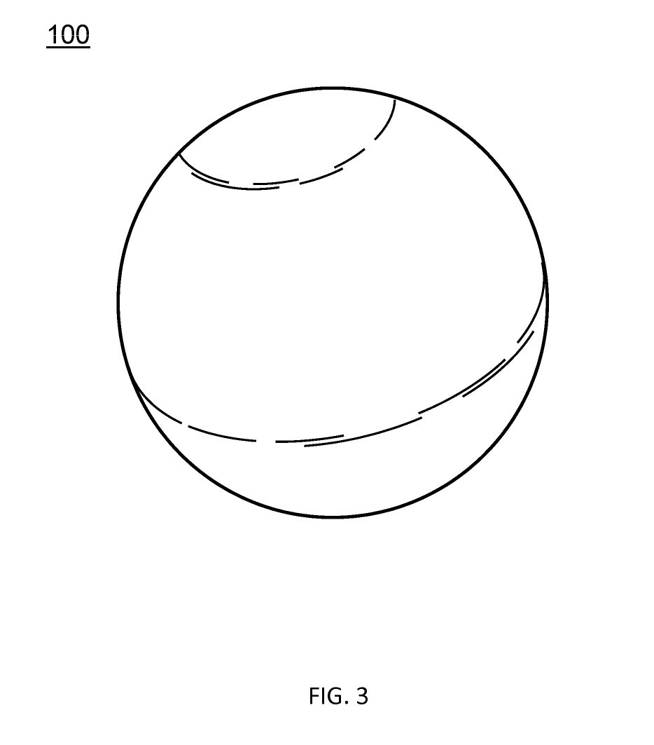

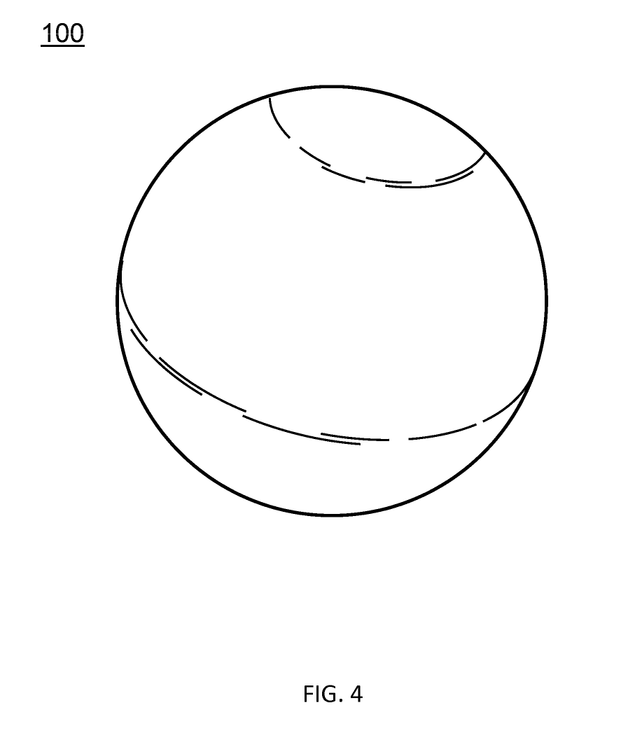

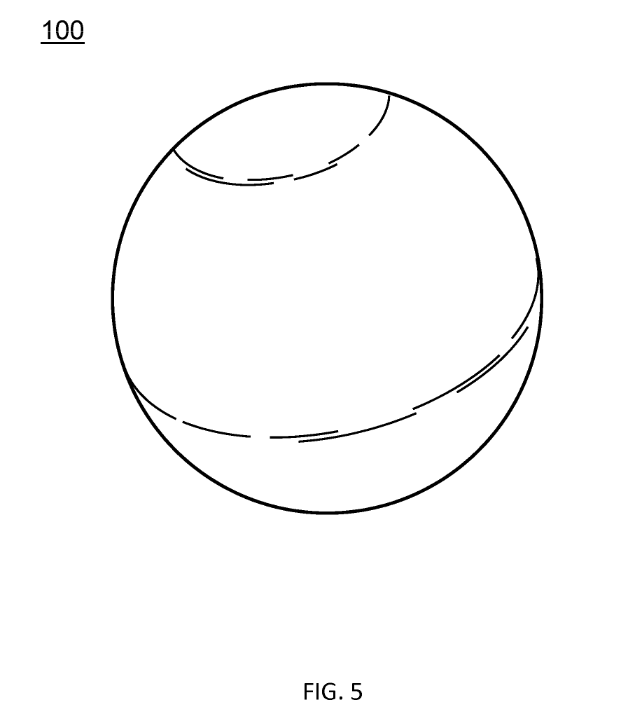

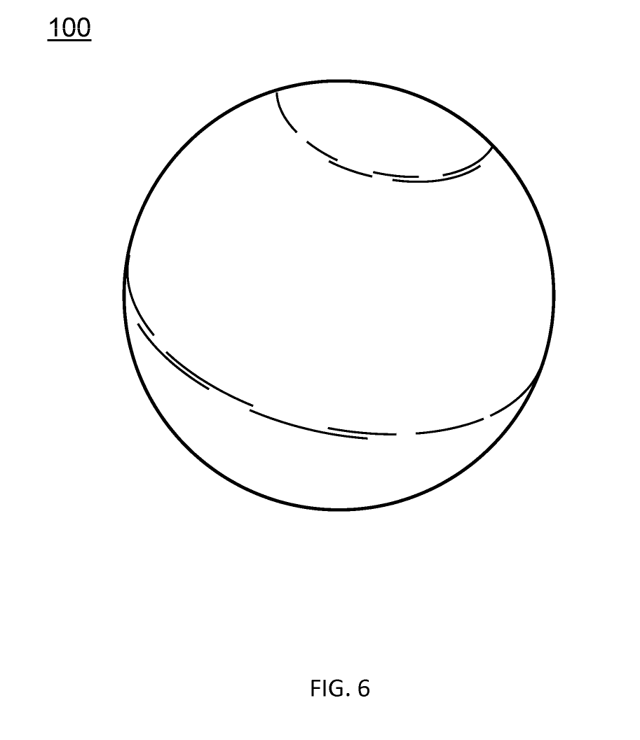

As shown in FIGS. 1 through 6 and as noted above, the present invention relates to a substantially spherically-shaped ear plug 100. As can be appreciated by one skilled in the art, although the ear plug 100 is described as sphere shaped, it does not need to exactly represent a sphere as small variations can be realized such that it is substantially spherically shaped yet still retains an inner and outer dome shape as described below.

The ear-plug 100 is formed of silicone to provide pliable and malleable properties. A non-limiting example of such a silicone compound is silanol terminated PDMS (also known as Polydimethyl Silicone).

Additionally, because the ear plug 100 is made of silicone, when it is positioned and depressed into the ear canal, the front end of the ear plug 100 is pushed out and deeper into the ear canal to create a safe seal. Finally, the tacky consistency of silicone creates an airtight/watertight seal.

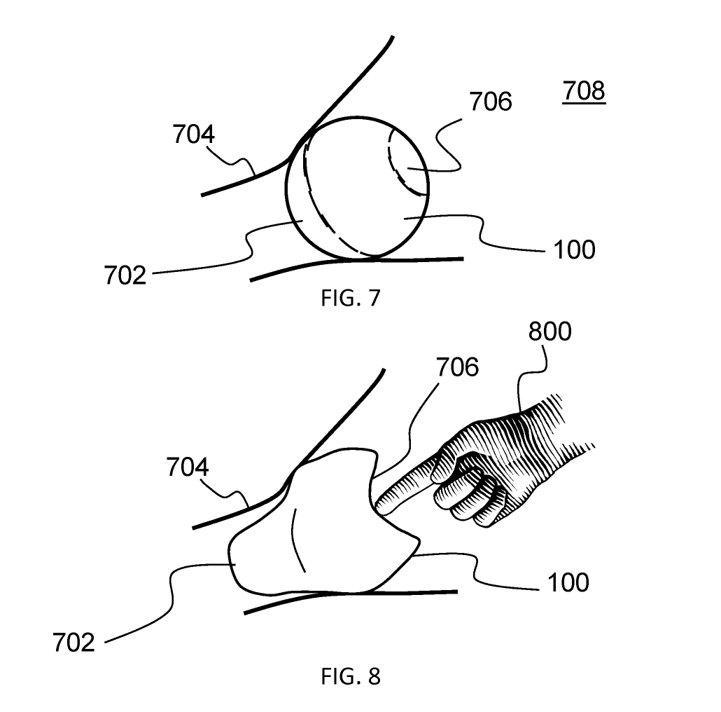

As shown in FIG. 7, because of the spherical shape, when the ear plug 100 is initially positioned into the ear, an inner dome shape 702 (front end) is positioned within the ear canal 704 while an opposing outer dome shape 706 is positioned toward outside 708 the ear canal 704.

As shown in FIG. 8, the outer dome shape 706 provides a surface for a user 800 to press into to force the ear plug 100 into the ear canal 704. Due to the spherical shape and silicone material, the inner dome shape 702 is expanded and bulges into the ear canal 704 when the outer dome shape 706 is compressed. Thus, once positioned into the ear canal 704, the tacky consistency of silicone creates an airtight/watertight seal between the ear plug 100 and ear canal 704.

It should be noted that silicone is a very sticky substance and, as such, is extremely difficult to mold into a precise shape. Because of the nature of silicone, existing silicone ear plugs are either extruded or stamped out from a sheet. Through such an extrusion or stamping process, the existing techniques for creating a silicon ear plug result in ear plugs that are not precisely shaped and, importantly, cannot be formed spherically.

As described above, a spherically-shaped ear plug 100 that is formed of silicone provides unique benefits that allow for compression of the ear plug 100 at the outer dome shape 706 to force the inner dome shape 702 into the ear canal 704. However, as mentioned, due to the nature of silicone and the difficulty of creating such a spherically-shaped ear plug 100 made of silicone, such an invention has not heretofore been created as existing techniques cannot be used to make such a device.

Thus, the present invention is also directed to a unique method for making the spherically-shaped ear plug 100. The method described herein was specifically devised in order to overcome the issues as presented by silicone and the multi-dimensional shape of a sphere.

It should be understood that although the method is described as being directed to a spherically-shaped ear plug 100, the invention with respect to the method is not limited thereto, as it can be applied to any suitably-shaped ear plug that is made of silicone.

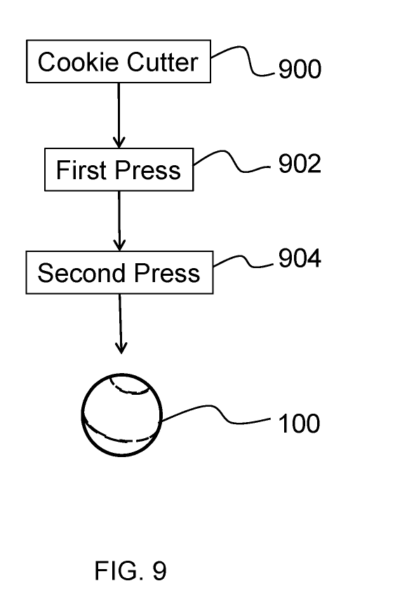

Generally speaking and as depicted in FIG. 9, the method for creating the spherically-shaped ear plug 100 includes three main steps, a cookie cutter step 900, a first press step 902, and a second press step 904. For clarity, each step will be described in turn below.

(1.1) Cookie Cutter Step

The cookie cutter step 900 is used to initially shape the silicone. In doing so, an uncured silicone compound (e.g., silanol terminated PDMS, also known as Polydimethyl Silicone) is milled into sheets that are cut into squares. The silicone sheets are rolled out onto a hard surface that is coated with polyurethane or another suitable substance. Stainless steel cookie cutters are placed onto the silicone sheet and carefully rolled with a weighted roller to force the silicone material into the cookie cutter.

The material is leveled off to the top of the cookie cutter. Care has to be taken in this process to not compress the material too much so as not to have excessive decompression over time, which would affect the end ear plug volume.

The cookie cutter filled with silicone material is placed in a custom slot (to hold it rigidly in place) into the first press (e.g., pneumatic press).

(1.2) First Press

The first press includes stainless steel pins that are forced down through the silicone in the cookie cutter. As a non-limiting example, 10 stainless steel pins can be used. Beneath the cookie cutter is a plate that has holes in it that are formed to exactly accommodate the pins that are present in the first press. The plate is formed of any suitable material that prevents corrosion, non-limiting examples of which include stainless steel or Delrin® acetal resin.

The holes and pins line up with the slots in an open tray (e.g., plastic tray) that is positioned below the first press. Although not limited thereto, the open tray can be used as the actual final packaging, with tray slots that are shaped in the desired shape of the final product. In this case, the tray slots in the open plastic tray are semi-spherically shaped to support and accommodate the spherically-shaped ear plug.

When the first press is activated, the pins are pushed through the silicone in the cookie cutter and through the holes in the plate to deposit the silicone “pills” into the bottom of the open plastic tray. The pins, when pushed through the cookie cutter, must be flush with the bottom of the plate or material residue will accumulate on the pins and in the plate.

The tray is housed in a polyurethane nest to give it proper support to receive the pills. The stainless steel pins have tiny holes drilled through their length. Air is forced through these holes just as they are driven through the silicone in the cookie cutter. The air pressure releases the silicone pills from the ends of the pins, preventing them from sticking and guiding the pills into the tray. Care must be taken not to use too much pressure or a dimple will form on the top of the plugs from where the air has hit the material at excessive pressure. The tray must have enough clearance from the bottom of the plate so that the pills will be almost touching the tray when they are released from the pins. If there is too much distance between tray and bottom of the plate, the pills will not align into the tray. Alternatively, if the tray is too close to the bottom of the plate, the pills will smash into the tray, thereby preventing proper shaping of the silicone in the second press. The pills must be of an exact height, diameter and volume so as to form the plug shape size as required. This is controlled by the pin diameter and cookie cutter depth.

It should be noted that the stainless steel and Delrin® materials (or similar materials) are critical in order to give strength to the parts and also to prevent corrosion/reaction to the silicone. The polyurethane assists in providing a substrate that will not stick to the silicone.

(1.3) Second Press

The open tray with pills is then moved to the second press. The tray is placed into the polyurethane nest in the second press. The second press includes a polyurethane mold at the top that is identical in shape to the top of the tray. In other words, the tray includes tray slots that are shaped in the desired shape of the final product, with the mold having mold slots that correspond to the shape of the tray slots. As a non-limiting example, the tray slots are semi-spherically shaped, with the mold slots also being semi-spherically shaped.

Below the mold is a protective release layer (e.g., nitrile disk) that is inserted into a slot in the press. The protective lease layer is a layer that acts as a barrier that is positioned between the silicon and mold. The protective release layer is important in that without the release layer, the silicone would stick in the mold. Immediately below the release layer is the nest holding the tray and pills. When the press is operated, the top mold is forced downward. At the same time, a vacuum is used to suck the release layer upward toward this top mold. It should be noted that during initial production, the release layer was causing some of the pills to be forced downward into the bottom of the mold. Thus, the vacuum was implemented to remedy this issue.

The mold and release layer are then pressed down on top of the tray with the pills sitting in the tray. The vacuum causes the release layer to be sucked up slightly into the top mold which allows the silicone pills to be formed more precisely into the shape of the mold and form a smoother surface. The press is released and the release layer springs back to its original shape (i.e., flat shape), releasing the molded silicone earplugs into the tray, thereby preventing any sticking of the material to the top mold or release layer. The tray with the uncured, molded and spherically-shaped silicone ear plugs is then inspected and snapped shut, ready for final packaging.

What is claimed is:

1. A spherically-shaped ear plug, comprising:
an ear plug formed of silicone, the ear plug being substantially spherically shaped.

2. A method for forming a shaped ear plug, comprising acts of:
using a cookie cutter to shape an uncured silicone compound, filling the cookie cutter with the silicone;
positioning the cookie cutter in a slot of a first press, the first press having pins with air holes therethrough;
driving the pins of the first press through the silicone in the cookie cutter to deposit silicone pills into tray slots of an open tray;
forcing air through the air holes as the pins are driven through the silicone to release the silicone pills from the pins and into the tray slots;
using a second press having a mold with mold slots, the press having a release layer attached thereto for positioning between the mold and silicon;
vacuuming the release layer into the mold;
pressing the mold and release layer onto the open tray to form the ear plugs; and
releasing the press to cause the release layer to spring to its original shape and release the ear plugs into the open tray.

3. The method as set forth in claim 2, wherein the tray slots of the open tray have a semi-spherical shape, with the mold slots having a corresponding semi-spherical shape.

4. A spherically-shaped ear plug formed according to the method of claim 2.

5. A spherically-shaped ear plug formed according to the method of claim 3.
